(19) United States
(12) Patent Application Publication
(10) Pub. No.: US 2024/0296908 A1
(43) Pub. Date: Sep. 5, 2024

(54) METHOD AND APPARATUS FOR IDENTIFYING FUSION GENE, DEVICE, PROGRAM AND STORAGE MEDIUM

(71) Applicants: Chengdu BOE Optoelectronics Technology Co., Ltd., Chengdu, Sichuan (CN); BOE TECHNOLOGY GROUP CO., LTD., Beijing (CN)

(72) Inventor: Mengjia Liu, Beijing (CN)

(73) Assignees: Chengdu BOE Optoelectronics Technology Co., Ltd., Chengdu, Sichuan (CN); BOE TECHNOLOGY GROUP CO., LTD., Beijing (CN)

(21) Appl. No.: 18/027,521

(22) PCT Filed: Mar. 28, 2022
(86) PCT No.: PCT/CN2022/083275

§ 371 Date: Mar. 21, 2023

(51) Int. Cl. G16B 30/00

(52) U.S. Cl. CPC G16B 30/00

(57) ABSTRACT

The present disclosure provides a method and apparatus for identifying a fusion gene, a device, a program and a storage medium, belonging to the technical field of gene detection. The method includes: acquiring a target gene sequencing sequence to be identified and a reference gene sequence; aligning the target gene sequencing sequence to the reference gene sequence, and acquiring distribution and targeted capturing results of spanning reads and split reads of the target sequencing sequence located in a target area; screening a target fusion gene pair from the reads based on the distribution and the targeted capture results; and outputting an identification result regarding the target fusion gene pair.

### FIELD

The present disclosure belongs to the technical field of gene detection and more particularly, relates to a method and apparatus for identifying a fusion gene, a device, a program and a storage medium.

### BACKGROUND

Gene fusion refers to a process by which chromosomal transposition, deletion or reversal causes all or part of sequences of two unrelated genes to fuse into a new gene. Tens of thousands of gene fusions have been discovered. At present, many gene fusions have been reported to be closely related to the occurrence of cancers, among which, Anaplastic Lymphoma Kinase (ALK), ROS1 (ROS proto-oncogene 1, receptor tyrosine kinase and c-ros sarcoma oncofactor-receptor tyrosine kinase), NeuroTrophin Receptor Kinase (NTRK), and other common fusion genes are used as diagnostic tools for certain cancers, and the like. According to the latest research reports, more than one thousand gene fusions have been identified, among which tumor driver gene fusion has become a hot spot in scientific research.

### SUMMARY

The present disclosure provides a method and apparatus for identifying a fusion gene, a device, a program and a storage medium.

The method includes:
acquiring a target gene sequencing sequence to be identified and a reference gene sequence;
aligning the target gene sequencing sequence to the reference gene sequence, and acquiring distribution and targeted capturing results of spanning reads and split reads of the target gene sequencing sequence located in a target area;
screening a target fusion gene pair from the reads based on the distribution and the targeted capture results; and
outputting an identification result regarding the target fusion gene pair.

In some embodiments of the present disclosure, the step of screening the target fusion gene pair from the reads based on the distribution and the targeted capture results includes:
calculating a breakpoint position of the read according to the number of the split reads and strongly supported split reads; and
screening the target fusion gene pair from the reads according to the breakpoint position and positions of the spanning reads.

In some embodiments of the present disclosure, the step of screening the target fusion gene pair from the read according to the breakpoint position and the positions of the spanning reads includes:
filtering a read that does not have the supported spanning read, at the upstream and downstream of the breakpoint position included in the read;
regarding the reads reserved after filtration as candidate fusion gene pairs in the case that the first end and the second end of the read reserved after filtration are located in different genes; and
filtering a low-quality fusion gene pair in the candidate fusion gene pairs to obtain the target fusion gene pair.

In some embodiments of the present disclosure, the step of filtering the low-quality fusion gene pair in the candidate fusion gene pairs to obtain the target fusion gene pair includes:
filtering paralogous genes in the candidate fusion gene pairs to obtain first candidate fusion gene pairs;
calculating a number of gene mappings contained in the first candidate fusion genes;
filtering the first candidate fusion gene pairs in which the number of the gene mappings is greater than or equal to a number threshold of gene mappings to obtain a second candidate fusion gene pair;
calculating a fusion gene score of the second candidate fusion gene pair according to a distance between the breakpoint positions of the second candidate fusion gene pair, and an average sequencing depth in the target area; and filtering the second candidate fusion gene pair whose fusion gene score is less than a
fusion gene score threshold to obtain the target candidate fusion gene pair.

In some embodiments of the present disclosure, the step of calculating the fusion gene score of the second candidate fusion gene pair according to the distance between the breakpoint positions of the second candidate fusion gene pair, and the average sequencing depth in the target area, includes:
solving a difference between a sum of the distances between the spanning read in the second fusion gene pair and two breakpoints, and a peak value of an insert length of a genome methylated sequencing sequence, as a first factor score;
regarding a ratio of distances between two ends of the spanning read in the second fusion gene pair and the breakpoint position to a length of the read as a second factor score;
regarding a ratio of distances between two ends of the split read in the second fusion gene pair and the breakpoint position to a multiplication length of the read as a third factor score, wherein the multiplication length is a product of the length of the read and a multiplication parameter; and
regarding a ratio of a sum of the first factor score, the second factor score and the third factor score to the average sequencing depth in the target area as the fusion gene score of the second candidate fusion gene pair.

In some embodiments of the present disclosure, the step of aligning the target gene sequencing sequence to the reference gene sequence, and acquiring the distribution and targeted capturing results of the spanning reads and split reads of the target sequencing sequence located in the target area includes:
aligning the target gene sequencing sequence to the reference gene sequence to obtain an alignment result; and screening the spanning reads from the targeted sequencing result based on the alignment result in spanning read screening conditions, and screening the split reads and the strongly supported split reads from the targeted sequencing result based on the alignment result in split read screening conditions.

In some embodiments of the present disclosure, the step of screening the spanning reads from the targeted sequencing result based on the alignment result in the spanning read screening conditions includes:
screening a spanning read that meets the following spanning read screening conditions at the same time from the reads:
a sum value obtained by summing a length of a left-end read, a length of a right-end read and a distance between the left-end read and the right-end read of the read is greater than a product of the lower quartile of a length of the read and a target parameter, wherein the target parameter is a parameter that controls a number of outputted mappings and degree of stringency;
neither the left-end read nor the right-end read in the reads has a similar sequence; and
multiple alignment values of the left-end read and the right-end read in the reads include a proper aligner characteristic value and a secondary alignment characteristic value, and does not include a segment unmapped characteristic value and a next segment unmapped characteristic value.

In some embodiments of the present disclosure, the step of screening the split reads and the strongly supported split reads in the targeted sequencing result based on the alignment result in the split read screening conditions, includes:
screening a split read that meets the following split read screening conditions at the same time from the reads:
an alignment length of each position in the read is greater than a length threshold, and the alignment length is greater than one-third of a total length of the read;
a sequence whose length of the read is the alignment length has no similar sequence in the alignment result; and
a quality value of a number of alignment times of the read is greater than or equal to a number of alignment times; and determining the split read that meets the above split read conditions, in the case that
alignment positions of the left-end read and the right-end read overlap, to be a strongly supported split read.

In some embodiments of the present disclosure, the step of calculating the breakpoint position of the read according to the number of the split reads and the number of the strongly supported split reads includes:
regarding a maximum value of a weighted sum of the number of the split reads and the number of the strongly supported split reads in the reads as the breakpoint position.

In some embodiments of the present disclosure, before acquiring the target gene sequencing sequence to be identified and the reference gene sequence, the method further includes:
counting a base number, base quality and base lengths in the obtained target gene sequencing sequence; and
identifying sequences to be filtered in the targeted gene sequencing sequence according to the base number, the base quality and the base lengths, and filtering the sequences to be filtered.

In some embodiments of the present disclosure, the step of identifying the linker sequences in the target gene sequencing sequence according to the number of the bases, the quality of the bases and the lengths of the bases includes:
regarding sequencing sequences whose base quality is a quality threshold, minimum base length is a base length threshold, and average quality value of the sequencing sequence is lower than the quality threshold, as the sequences to be filtered; and
supplementing a sequencing sequence containing a left-end sequencing sequence or a right-end sequencing sequence whose overlap degree with the linker sequence reaching a preset degree, to the sequences to be filtered.

Some embodiments of the present disclosure provide an apparatus for identifying a fusion gene. The apparatus includes:
an acquisition module configured to acquire a target gene sequencing sequence to be identified and a reference gene sequence;
an alignment module configured to align the target gene sequencing sequence to the reference gene sequence, and acquiring distribution and targeted capturing results of spanning reads and split reads of the target gene sequencing sequence located in a target area;
a screening module configured to screen a target fusion gene pair from the reads based on the distribution and the targeted capture results; and
an outputting module configured to output an identification result regarding the target fusion gene pair.

In some embodiments of the present disclosure, the screening module is further configured for:
calculating a breakpoint position of the read according to the number of the split reads and strongly supported split reads; and
screening the target fusion gene pair from the reads according to the breakpoint position and positions of the spanning reads.

In some embodiments of the present disclosure, the screening module is further configured for:
filtering a read that does not have the supported spanning read, at the upstream and downstream of the breakpoint position included in the read;
regarding the reads reserved after filtration as candidate fusion gene pairs in the case that the first end and the second end of the read reserved after filtration are located in different genes; and
filtering a low-quality fusion gene pair in the candidate fusion gene pairs to obtain the target fusion gene pair.

In some embodiments of the present disclosure, the screening module is further configured for:
filtering paralogous genes in the candidate fusion gene pairs to obtain first candidate fusion gene pairs;
calculating a number of gene mappings contained in the first candidate fusion genes;
filtering the first candidate fusion gene pairs in which the number of the gene mappings is greater than or equal to a number threshold of gene mappings to obtain a second candidate fusion gene pair;
calculating a fusion gene score of the second candidate fusion gene pair according to a distance between the breakpoint positions of the second candidate fusion gene pair, and an average sequencing depth in the target area; and
filtering the second candidate fusion gene pair whose fusion gene score is less than a fusion gene score threshold to obtain the target candidate fusion gene pair.

In some embodiments of the present disclosure, the screening module is further configured for:
solving a difference between a sum of the distances between the spanning read in the second fusion gene pair and two breakpoints, and a peak value of an insert length of a genome methylated sequencing sequence, as a first factor score;
regarding a ratio of distances between two ends of the spanning read in the second fusion gene pair and the breakpoint position to a length of the read as a second factor score;
regarding a ratio of distances between two ends of the split read in the second fusion gene pair and the breakpoint position to a multiplication length of the read as a third factor score, wherein the multiplication length is a product of the length of the read and a multiplication parameter; and
regarding a ratio of a sum of the first factor score, the second factor score and the third factor score to the average sequencing depth in the target area as the fusion gene score of the second candidate fusion gene pair.

In some embodiments of the present disclosure, the alignment module is further configured for:
aligning the target gene sequencing sequence to the reference gene sequence to obtain
an alignment result; and
screening the spanning reads from the targeted sequencing result based on the alignment result in spanning read screening conditions, and screening the split reads and the strongly supported split reads from the targeted sequencing result based on the alignment result in split read screening conditions.

In some embodiments of the present disclosure, the alignment module is further configured for:
screening a spanning read that meets the following spanning read screening conditions at the same time from the reads;
a sum value obtained by summing a length of a left-end read, a length of a right-end
read and a distance between the left-end read and the right-end read of the read is greater than a product of the lower quartile of a length of the read and a target parameter, wherein the target parameter is a parameter that controls a number of outputted mappings and degree of stringency;
neither the left-end read nor the right-end read in the reads has a similar sequence; and multiple alignment values of the left-end read and the right-end read in the reads
include a proper aligner characteristic value and a secondary alignment characteristic value, and does not include a segment unmapped characteristic value and a next segment unmapped characteristic value.

In some embodiments of the present disclosure, the alignment module is further configured for:
screening a split read that meets the following split read screening conditions at the same time from the reads:
an alignment length of each position in the read is greater than a length threshold, and the alignment length is greater than one-third of a total length of the read;
a sequence whose length of the read is the alignment length has no similar sequence in
the alignment result; and
a quality value of a number of alignment times of the read is greater than or equal to a number of alignment times; and
determining the split read that meets the above split read conditions, in the case that alignment positions of the left-end read and the right-end read overlap, to be a strongly supported split read.

In some embodiments of the present disclosure, the screening module is further configured for:
regarding a maximum value of a weighted sum of the number of the split reads and the number of the strongly supported split reads in the reads as the breakpoint position.

In some embodiments of the present disclosure, the acquisition module is further configured for:
counting a base number, base quality and base lengths in the obtained target gene sequencing sequence; and identifying sequences to be filtered in the targeted gene sequencing sequence according
to the base number, the base quality and the base lengths, and filtering the sequences to be filtered.

In some embodiments of the present disclosure, the acquisition module is further configured for:
regarding sequencing sequences whose base quality is a quality threshold, minimum
base length is a base length threshold, and average quality value of the sequencing sequence is lower than the quality threshold, as the sequences to be filtered; and
supplementing a sequencing sequence containing a left-end sequencing sequence or a right-end sequencing sequence whose overlap degree with the linker sequence reaching a preset degree, to the sequences to be filtered.

Some embodiments of the present disclosure provide a computing processing device, including:
a memory, configured to store a computer-readable code therein; and
one or more processors, when the computer-readable code is executed by the one or more processors, causes the computing processing device to execute the method for identifying the fusion gene.

Some embodiments of the present disclosure provide a computer program, including a computer-readable code, which when being operated on the computing processing device, causes the computing processing device to execute the method for identifying the fusion gene.

Some embodiments of the present disclosure provide a non-transitory computer-readable medium, in which the method for identifying the fusion gene as described above is stored.

The above description is only an overview of the technical solution of the present disclosure. In order to better understand the technical means of the present disclosure, it can be implemented according to the contents of the specification, and in order to make the above and other purposes, features and advantages of the present disclosure more obvious and understandable, the specific implementation methods of the present disclosure are listed below.

### BRIEF DESCRIPTION OF THE DRAWINGS

In order to more clearly explain the embodiments of the present disclosure or the technical solutions in the related art, the following will briefly introduce the drawings needed in the embodiments or the description of the related art. It is obvious that the drawings in the following description are some embodiments of the present disclosure. For those skilled in the art, other drawings can also be obtained from these drawings without creative work.

FIG. 1 schematically shows a schematic flow chart of a method for identifying a fusion gene according to some embodiments of the present disclosure;

FIG. 2 schematically shows a schematic flow chart I of another method for identifying a fusion gene according to some embodiments of the present disclosure:

FIG. 3 schematically shows a schematic flow chart II of another method for identifying a fusion gene according to some embodiments of the present disclosure:

FIG. 4 schematically shows a schematic flow chart III of another method for identifying a fusion gene according to some embodiments of the present disclosure:

FIG. 5 schematically shows a schematic diagram I of a principle of another method for identifying a fusion gene according to some embodiments of the present disclosure:

FIG. 6 schematically shows a schematic flow chart IV of another method for identifying a fusion gene according to some embodiments of the present disclosure:

FIG. 7 schematically shows a schematic diagram II of a principle of another method for identifying a fusion gene according to some embodiments of the present disclosure:

FIG. 8 schematically shows a schematic diagram III of a principle of another method for identifying a fusion gene according to some embodiments of the present disclosure:

FIG. 9 schematically shows a schematic diagram IV of a principle of another method for identifying a fusion gene according to some embodiments of the present disclosure:

FIG. 10 schematically shows a schematic diagram V of a principle of another method for identifying a fusion gene according to some embodiments of the present disclosure:

FIG. 11 schematically shows a schematic diagram VI of a principle of another method for identifying a fusion gene according to some embodiments of the present disclosure:

FIG. 12 schematically shows a schematic diagram I of an effect of another method for identifying a fusion gene according to some embodiments of the present disclosure:

FIG. 13 schematically shows a schematic diagram II of an effect of another method for identifying a fusion gene according to some embodiments of the present disclosure:

FIG. 14 schematically shows a schematic diagram III of an effect of another method for identifying a fusion gene according to some embodiments of the present disclosure:

FIG. 15 schematically shows a schematic diagram IV of an effect of another method for identifying a fusion gene according to some embodiments of the present disclosure:

FIG. 16 schematically shows a schematic diagram V of an effect of another method for identifying a fusion gene according to some embodiments of the present disclosure:

FIG. 17 schematically shows a schematic diagram VI of an effect of another method for identifying a fusion gene according to some embodiments of the present disclosure:

FIG. 18 schematically shows a schematic diagram VII of an effect of another method for identifying a fusion gene according to some embodiments of the present disclosure:

FIG. 19 schematically shows a schematic diagram VIII of an effect of another method for identifying a fusion gene according to some embodiments of the present disclosure:

FIG. 20 schematically shows a schematic diagram IX of an effect of another method for identifying a fusion gene according to some embodiments of the present disclosure:

FIG. 21 schematically shows a schematic structural diagram of an apparatus for identifying a fusion gene according to some embodiments of the present disclosure:

FIG. 22 schematically shows a block diagram of a computing processing device for performing the method according to some embodiments of the present disclosure; and

FIG. 23 schematically shows a memory unit configured to save or carry a program code implementing the method in some embodiments of the present disclosure.

### DETAILED DESCRIPTION

In order to make the purpose, technical scheme and advantages of the embodiments of the present disclosure clearer, the technical solution in the embodiments of the present disclosure will be described clearly and completely in combination with the drawings in the embodiments of the present disclosure. Obviously, the described embodiments are part of the embodiments of the present disclosure, not all of them. Based on the embodiments in the present disclosure, all other embodiments obtained by ordinary technicians in the art without creative work fall within the scope of protection of the present disclosure.

With the development of precision medicine in the future, molecular diagnosis methods for identifying gene fusion will become an inevitable trend. In related art, gene fusion is mainly identified through two sequencing methods: whole genome sequencing (WGS) and transcriptome sequencing technology (RNA-seq), which have various advantages and disadvantages compared with conventional fluorescence in situ hybridization and other methods, see Table 1 for details. Fluorescence in situ hybridization and other methods have the characteristics of low throughput, but usually tumor sample materials are usually limited, so it is difficult to detect a plurality of fusions in low-throughput methods. WGS and RNA-seq methods have the characteristics of high throughput, but result in the difficulties in storage and computation of subsequent analysis of server resources and long analysis time due to high sequencing cost and large data volume. As targeted sequencing has gradually become a mainstream detection method in tumor diagnosis, early cancer screening, reproductive genetics, immunotherapy, etc., it is necessary to establish tools, analysis methods, and analysis processes for gene fusion identification with targeted sequencing.

At present, the existing methods of targeted sequencing for the identification of gene fusions have certain limitations, and most of these methods are based on the results of existing fusion genes to identify a group of gene fusions or a fusion gene for a cancer. It can be seen that these identification methods are to design probes only for fusion genes to identify fusions. In addition, most methods can only be applied to known fusion genes, but cannot identify unknown fusion genes. Conventional targeted sequencing is based on a target region design probe, which is configured to identify somatic/germ cell mutations, gene fusion, copy number variations, chromosome large segment variations, tumor mutation burden, tumor microsatellite instability and the like in a target interval. Therefore, the above detection methods have limitations in practical use and are not suitable for the identification of possible fusion genes in various types of conventional target intervals.

TABLE 1

| Detection | Application | | |
|---|---|---|---|
| method | scenarios | Advantages | Disadvantages |
| Fluorescence | Identify known | Long technology | Low throughput and dependency on |
| in situ | fusions | development time and | experience of an inspector |
| hybridization | occurring at | mature technology | Only suitable for analysis of known |
| | genomic level | | fusions |
| | | | Smaller chromosomal rearrangements |
| | | | are not easily detected out |
| | | | Transcripts produced by fusion genes |
| | | | cannot be distinguished |
| | | | Low throughput, allowing only one |
| | | | group or a few groups of fusions to be |
| | | | detected in a single detection |
| Whole | Identify | Identification results | 1. The sequencing cost is high |
| genome | various fusions | are complete, and | 2. The data volume for sequencing is |
| sequencing | occurring at | gene fusions over the | large, and the data storage and data |
| | genomic level | whole gene can be | processing requirements for a |
| | | identified | computer are high |
| | | | 3. The analysis time is long, and it |
| | | | takes a lot of time in the process of |
| | | | data preprocessing and alignment |
| RNA-seq | Identify gene | Analyze transcripts | 1. Only encoded gene fusions can be |
| | fusions | formed by different | analyzed |
| | occurring at | gene fusions | 2. The sequencing cost is high |
| | transcriptome | | |
| | level | | |
| Targeted | Target | High flexibility, | The requirements for targeted |
| sequencing | identification | determine Panel | customization are high, and targeted |
| | of fusion in a | according to | sequencing Panel design is required |
| | target area | experimental needs; | |
| | | affordable price, | |
| | | high depth, | |
| | | high-volume | |
| | | screening methods | |
| | | recognized by the | |
| | | market | |

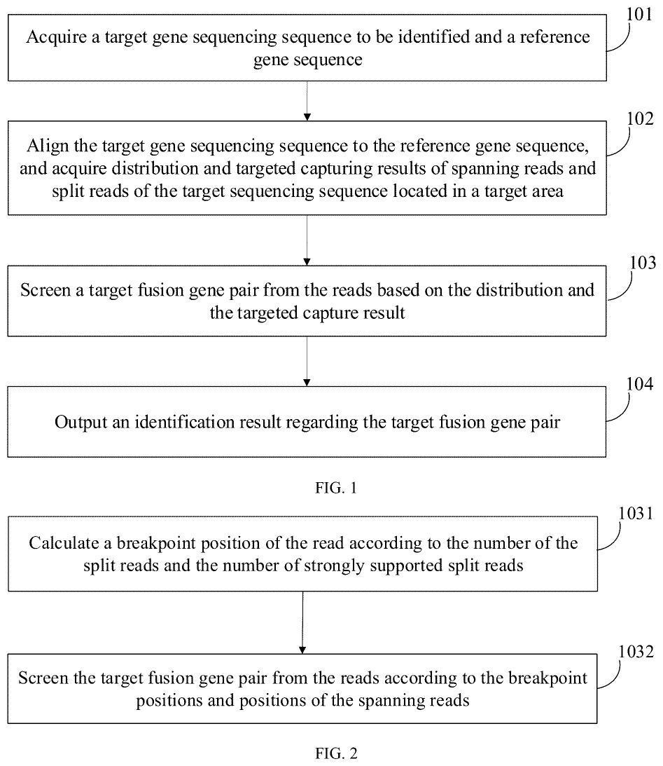

FIG. 1 schematically shows a schematic flow chart of a method for identifying a fusion gene according to the present disclosure. The method includes the following steps.
Step 101: Acquire a target gene sequencing sequence to be identified and a reference gene sequence.

In an embodiment of the present disclosure, the target gene sequencing sequence to be identified is a gene sequencing sequence collected by targeted sequencing of a sample genome in upstream experiments. The method of targeted sequencing may refer to commonly used targeted sequencing methods in the art. The method of targeted sequencing is not the focus of the present disclosure, and will not be repeated herein. The reference gene sequence is a genome sequencing sequence obtained by genetic sequencing of high-quality human genomes.

In some embodiments of the present disclosure, after acquiring the targeted sequencing data, low-quality data in the targeted sequencing sequence may be filtered by means of preprocessing. This preprocessing method may refer to, for example, linker sequence removal, low-quality sequence filtration and other methods which can be set according to actual needs, and not limited herein.
Step 102: Align the target gene sequencing sequence to the reference gene sequence, and acquire distribution and targeted capturing results of spanning reads and split reads of the target gene sequencing sequence located in a target area.

In an embodiment of the present disclosure, a spanning read refers to a read which covers a fusion site, and a left-end read and a right-end read of which may be aligned to different genes; and a split read refers to a read that happens to be at the fusion site. The left-end read and the right-end read refer to two fragments at opposite ends of the read, respectively. Specifically, the division of the left-end read and the right-end read may be determined according to the arrangement of sequences in the read. The left and right directions are different due to different arrangement. For the alignment of the target gene sequencing sequence to the reference gene sequence, in the case of human DeoxyriboNucleic Acid (DNA), a reference gene sequence of Hg19 or CRCh38 version may be selected as a sample; and BWA MEN may be selected as an alignment tool, and after the alignment is completed, the alignment results may be linearly sorted in an order of the reference genome and stored in a bam format. Then, the distribution and the target capturing results of reads that are successfully mapped in the target sequencing sequence in the target area are calculated according to a mapping relationship between a Read1 sequence and a Read2 sequence sequenced in double-ended sequencing in the alignment result; and the target capturing results refers to positions of respective reads in the target area for subsequent identification of fusion genes.
Step 103: Screen a target fusion gene pair from the reads based on the distribution and the targeted capture results.

In an embodiment of the present disclosure, considering that the distributions and positions of the spanning read and the split read in the fusion gene pair are significantly different from those of a non-fusion gene, in the present disclosure, the respective reads in the target area are screened according to the number, base proportion and the characteristics of distribution of the spanning reads and the split reads in the respective reads in the target area and a screening rule that is formulated by using position characteristics of the spanning reads and the split reads. Therefore, a target fusion gene pair that meets the distribution characteristics of the spanning reads and the split reads in the fusion gene pair is screened from the successfully mapped gene pairs. In this way, different screening conditions can be formulated for different identification needs to identify the fusion gene pairs, eliminating the need to develop dedicated targeted sequencing methods for specific fusion gene species. The identified fusion genes are no longer limited to fusion gene species targeted by a targeted sequencing method, which improves the utilization rate of targeted sequencing data.
Step 104: Output an identification result regarding the target fusion gene pair.

In an embodiment of the present disclosure, after the target fusion gene pair is identified, in order to allow the user to virtually view the identification result, the target fusion gene pair in the targeted sequencing result may be processed through a visualization module, which may be IGV, Read Map and other functional programs for visual output of gene sequencing data. Further, the distribution and the targeted capturing results of the spanning reads and the split reads and other data collected during the identification process of fusion genes may also be displayed as identification results together, so that users can verify and correct the identification results.

In an embodiment of the present disclosure, by performing fusion gene identification for conventional targeted sequencing and aligning the reference gene sequence to the target area of targeted sequencing, the distribution and target capture results of the split reads and spanning reads near the target area are obtained. The fusion gene pairs in the target gene sequencing sequence are screened out by using the distribution characteristics of the split reads and the spanning reads in the fusion gene pairs. The screening may be performed against the distribution of the reads in different fusion genes, so that the fusion gene identification results of the target gene sequencing sequence are no longer limited to specific fusion gene species, which increases the utilization rate of the targeted gene sequencing data in the gene fusion identification.

In some embodiments, referring to FIG. 2, the step 103 includes the following steps.
Step 1031: Calculate a breakpoint position of the read according to the number of the split reads and the number of strongly supported split reads.

In an embodiment of the present disclosure, the strongly supported split read refers to a split read in which alignment positions of a left-end read and a right-end read are overlapped on a genome. The breakpoint position of the read refers to a position of a gene breakpoint of the fusion gene pair due to gene transposition, substitution or other reasons. The breakpoint position may be located based on the number of the identified split reads and the number of the strongly supported split reads.
Step 1032: Screen the target fusion gene pair from the reads according to the breakpoint position and positions of the spanning reads.

In an embodiment of the present disclosure, since a read with a breakpoint is not necessarily a fusion gene pair, it is necessary to further screen out the fusion gene pair from the read with the breakpoints according to the breakpoint position in the read and the position distribution of the spanning reads.

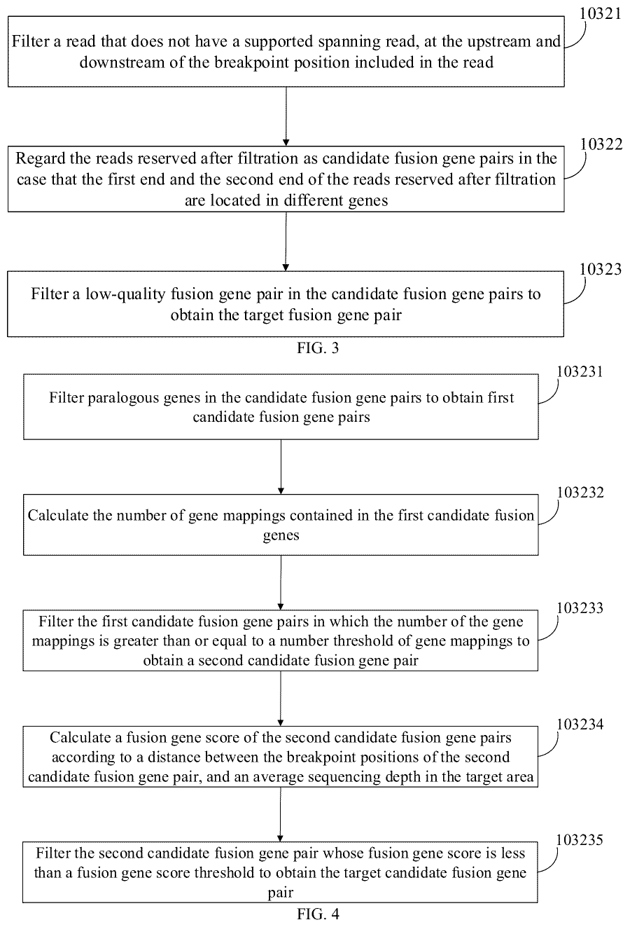

In some embodiments, referring to FIG. 3, the step 1032 includes the following steps.
Step 10321: Filter a read that do not have a supported spanning read, at the upstream and downstream of the breakpoint position included in the read.

In an embodiment of the present disclosure, in the case that there is no spanning read within a length range of lower quartile data of the read length at the front and back ends of the breakpoint position, this read is discarded. In the case that there is a spanning read within a length range of lower quartile data of the read length at the front and back ends of the breakpoint position, the breakpoint position is considered to be reliable and this read is reserved for subsequent further analysis.
Step 10322: Regard the reads reserved after filtration as candidate fusion gene pairs in the case that the first end and the second end of the read reserved after filtration are located in different genes.

In an embodiment of the present disclosure, the first end and the second end refer to two ends opposite in position of the read. The first end and the second end, that is, the left end and the right end, of the read at the identified breakpoint position are annotated. That is, a gene position where the left end and the right end are located is identified by using a GFF3 format file corresponding to a genomic version. Currently, the reads are used as candidate fusion pairs only when the left end and the right end are in different genes.
Step 10323: Filter a low-quality fusion gene pair from the candidate fusion gene pairs to obtain the target fusion gene pair.

In an embodiment of the present disclosure, the target fusion gene pair is further screened from the identified candidate fusion gene pairs through quality evaluation criteria to ensure the quality of the outputted target fusion gene pair. The quality evaluation criteria may be formulated through quality score, credibility score, data accuracy and other parameters, which may be specifically set according to actual needs, and are not limited here.

In some embodiments, referring to FIG. 4, the step 10323 includes the following steps.
Step 103231: Filter paralogous genes in the candidate fusion gene pairs to obtain first candidate fusion gene pairs.

In an embodiment of the present disclosure, the paralogous genes are genes derived from gene duplication in the same species that may evolve new functions that are related to the original functions. For the identified candidate fusion genes, whether the fusion genes are the paralogous genes are identified. When the fusion genes are the paralogous genes, candidate fusion gene pairs in this combination are filtered, which are not considered as the fusion gene pairs; and in the case of no paralogous genes, they are reserved as the first candidate fusion gene pairs for further filtration.
Step 103232: Calculate the number of gene mappings contained in the first candidate fusion genes.
Step 103233: Filter the first candidate fusion gene pairs in which the number of the gene mappings is greater than or equal to a number threshold of gene mappings to obtain a second candidate fusion gene pair.

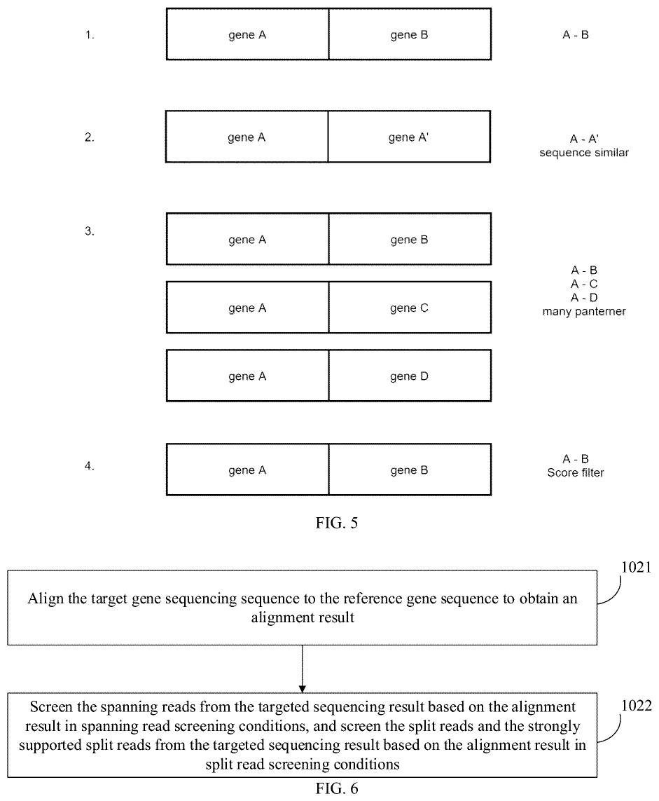

In an embodiment of the present disclosure, for the identified first candidate fusion genes, the number of mappings of the first candidate fusion genes is calculated. Referring to FIG. 5, in the case that geneA is mapped with geneB, geneC, and geneD at the same time, this combination is filtered out and is not considered to be fusion genes, otherwise it is used as the second candidate fusion gene pair. Certainly, this is just an exemplary illustration, and the number threshold of the gene mappings here is 3. The number threshold of the gene mappings may also be other positive integers greater than 1, which may be set according to actual needs, and not limited herein.
Step 103234: Calculate a fusion gene score of the second candidate fusion gene pair according to a distance between the breakpoint positions in the second candidate fusion gene pair, and an average sequencing depth in the target area.
Step 103235: Filter the second candidate fusion gene pair whose fusion gene score is less than a fusion gene score threshold to obtain the target candidate fusion gene pair.

In an embodiment of the present disclosure, a confidence level of the fusion gene pair may be measured according to the fusion gene score, and the fusion gene score is calculated by a fusion gene score calculation formula which is set according to a distance between adjacent breakpoint positions and the average sequencing depth of the target area. For example, the higher the fusion gene score is, the higher the confidence level of the fusion gene is. The fusion gene score threshold may be adjusted by parameters, the larger the fusion gene score threshold, the higher the confidence level of the fusion gene pair, which may be set according to actual needs, and not limited herein.

In an optional embodiment, the step 103234 may include the following steps:
Step N1, solving a difference between a sum of the distances between the spanning read in the second fusion gene pair and two breakpoints and a peak value of an insert length of a genome methylated sequencing sequence, as a first factor score;
Step N2, regarding a ratio of distances between two ends of the spanning read in the second fusion gene pair and the breakpoint position to a length of the read as a second factor score;
Step N3, regarding a ratio of distances between two ends of the split read in the second fusion gene pair and the breakpoint position to a multiplication length of the read as a third factor score, wherein the multiplication length is a product of the length of the read and a multiplication parameter; and
Step N4, regarding a ratio of a sum of the first factor score, the second factor score and the third factor score to the average sequencing depth in the target area as the fusion gene score of the second candidate fusion gene pair.

In some embodiments, referring to FIG. 6, the step 102 includes the following steps:
Step 1021, align the target gene sequencing sequence to the reference gene sequence to obtain an alignment result; and
Step 1022, screen the spanning reads from the targeted sequencing result based on the alignment result in spanning read screening conditions, and screen the split reads and the strongly supported split reads from the targeted sequencing result based on the alignment result in split read screening conditions.

In an embodiment of the present disclosure, the length of each read in the alignment result, as well as lengths of a right-end read and a left-end read of each read, a distance of the left-end read and the right-end read on a genome and other parameter indexes are measured according to the alignment result. Then, the spanning read, the split read and the strongly supported split read in the split read in the targeted sequencing result are determined by screening the respective reads according to the calculated parameter indexes of the reads as well as the spanning read screening conditions and the split read screening conditions.

In an optional embodiment, the step 1022 includes the following steps S1 to S3.
Step S1: Screen a spanning read that meets the following spanning read screening conditions A1 to A3 is screened from the reads:
A1: a sum value obtained by summing a length of the left-end read, a length of the right-end read and a distance between the left-end read and the right-end read is greater than a product of the lower quartile of the length of the read and a target parameter, and the target parameter being a parameter that controls the number of outputted mappings and the degree of stringency;

in an embodiment of the present disclosure, the spanning read needs to meet the following equation (1):

$$d + {L}_{1} + {L}_{2} > {Insert}_{d} \times C \qquad ( 1 )$$

in which d represents the distance between the left-end read R1 and the right-end read R2 in the read on the genome: L1 represents the length of the left-end read R1: L2 represents the length of the right-end read R2: Insert_{d} represents the lower quartile of the length of the read: C represents a parameter that controls the number of outputted mappings and the degree of stringency, which may be adjusted according to specific needs, and may be a positive integer ranging from 10 to 100.
A2: Neither the left-end read nor the right-end read in the read has a similar sequence.

In an embodiment of the present disclosure, the similar sequence refers to a sequence in which a homologous alignment result of two sequencing sequences is greater than a homologous alignment result threshold, and the homologous alignment result threshold may be set according to actual needs, and not limited herein. Referring to FIG. 7, neither the left-end read nor the right-end read of the spanning read has a plurality of similar sequences on a genome. That is, there is no similarity sequence in which the homologous alignment result is greater than 5, 10, 15 or other alignment result threshold.
A3: Multiple alignment values of the left-end read and the right-end read in the reads include a proper aligner characteristic value and a secondary alignment characteristic value, and does not include a segment unmapped characteristic value and a next segment unmapped characteristic value.

In an embodiment of the present disclosure, multiple alignment values of any of the left-end read and the right-end read of the spanning read include only the proper aligner characteristic value and the secondary alignment characteristic value, and does not include the segment unmapped characteristic value and the next segment unmapped characteristic value. It should be noted that the next segment unmapped characteristic value is that the current mapped read for a sequencing band is not aligned to the next mapped segment.

In the present disclosure, the spanning read is screened by the set spanning read screening conditions, and the spanning read may be screened efficiently from the reads, thereby improving the efficiency of fusion gene identification.
Step S2: Screen a split read that meets the following split read screening conditions B1 to B4 from the reads:
B1: an alignment length of each position in the read is greater than a length threshold, and the alignment length is greater than one-third of a total length of the read;
in an embodiment of the present disclosure, the split read needs to meet the following equation (2):

$$N > 20 and N > L / 3 \qquad ( 2 )$$

in which, N represents the alignment length of each position in the read; 20 refers to the length threshold; and L represents the total length of the read. Certainly, Formula (2) here is only an exemplary illustration, and specific N and L may be set according to actual needs, and not limited herein.
B2: A sequence whose length of the read is the alignment length has no similar sequence in the alignment result.

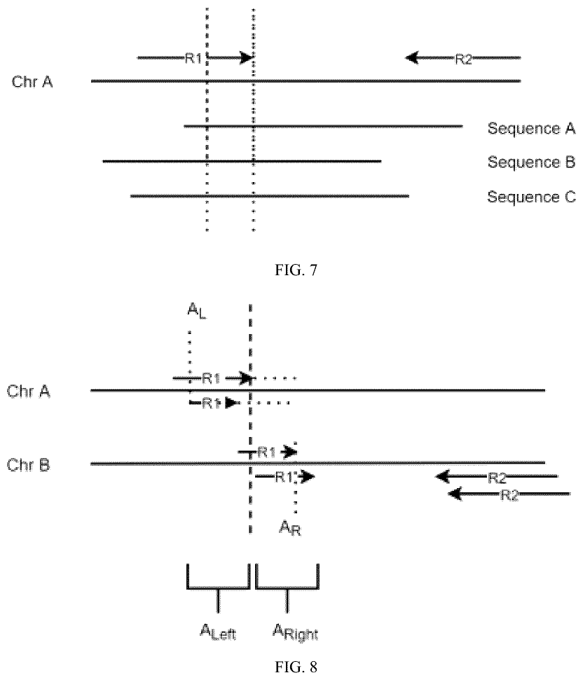

In an embodiment of the present disclosure, referring to FIG. 8, the sequence having an alignment length N do not have many similar sequences on a genome. That is, there is no similarity sequence in which the homologous alignment result is greater than 5, 10, 15 or other alignment result threshold.
B3: A quality value of the number of alignment times of the reads is greater than or equal to a number of alignment times.

In an embodiment of the present disclosure, the quality value Q of the number of alignment times is greater than or equal to the number of alignment times corresponding to 30, and is greater than 1.

In the present disclosure, the split read is screened by the set split read screening conditions, and the split read may be screened efficiently from the reads, thereby improving the efficiency of fusion gene identification.
Step S3: Determine the split read that meets the above split read conditions, in the case that alignment positions of the left-end read and the right-end read overlap, to be a strongly supported split read.

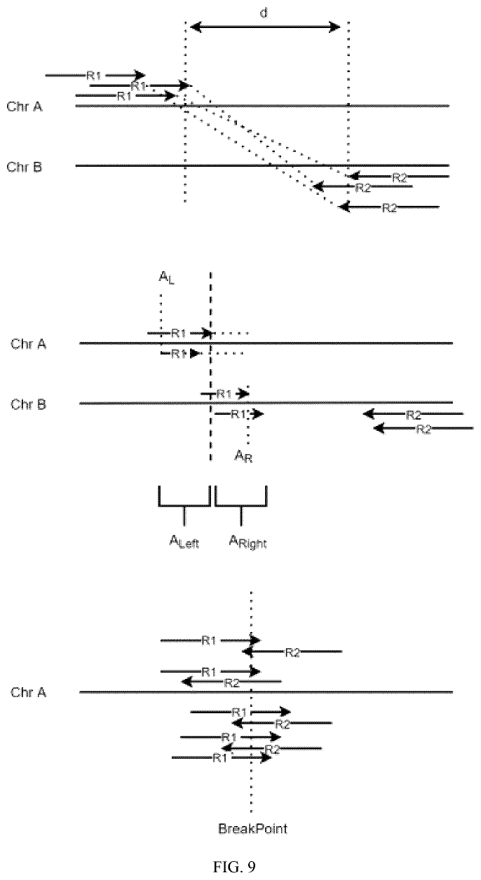

In an embodiment of the present disclosure, referring to FIG. 9, spanning read is seen in the top figure: split read is seen in the middle figure and the bottom figure: in the middle figure, R1 and R2 have no overlap; and in the bottom figure, R1 and R2 have an overlap. In the case that the alignment positions of the left-end read and the right-end read in the split read screened according to the conditions B1 to B3 have an overlap, i.e., the sequencing areas overlap, this alignment is labeled as the strongly supported split read in the split reads.

In an optional embodiment, the step 1031 includes: regarding a maximum value of a weighted sum of the number of the split reads in the reads and the number of the strongly supported split reads as the breakpoint position.

In an embodiment of the present disclosure, since the breakpoint position is generally related to the number of the split reads and the number of the strongly supported split reads, but some reads may have a plurality of split reads and strongly supported split reads, a maximum value of the number of the split reads and a maximum value of the number of the strongly supported split reads may be solved by assigning different weight values respectively to the number of the split reads and the number of the strongly supported split reads in the case of determining the breakpoint position, so as to determine the breakpoint position.

Specifically, the breakpoint position of the read may be calculated according to the following Formula (3):

$$I i = Max ( n * bi + m * Bi ) \qquad ( 3 )$$

in which, li represents the breakpoint position of the i^{th} read: bi represents the number of the split reads in the i^{th} read: Bi represents the number of the strongly supported reads in the i^{th} read: n represents a weight value of the split read; and m represents a weight value of the strongly supported read.

In an exemplary embodiment, in the case that the weight value n of the split read is 0.8 and the weight value m of the strongly supported split read is 2.5, the breakpoint position is Max (0.8bi+2.5Bi); or in the case that the weight value n of the split read is 0.6 and the weight value m of the strongly supported split read is 3, the breakpoint position is Max (0.6bi+3Bi); or in the case that the weight value n of the split read is 7 and the weight value m of the strongly supported split read is 10, the breakpoint position is Max (7bi+10Bi). Certainly, this is only an exemplary illustration, and the weight values of the split read and the strongly supported split read may be specifically set according to actual needs, and not limited herein.

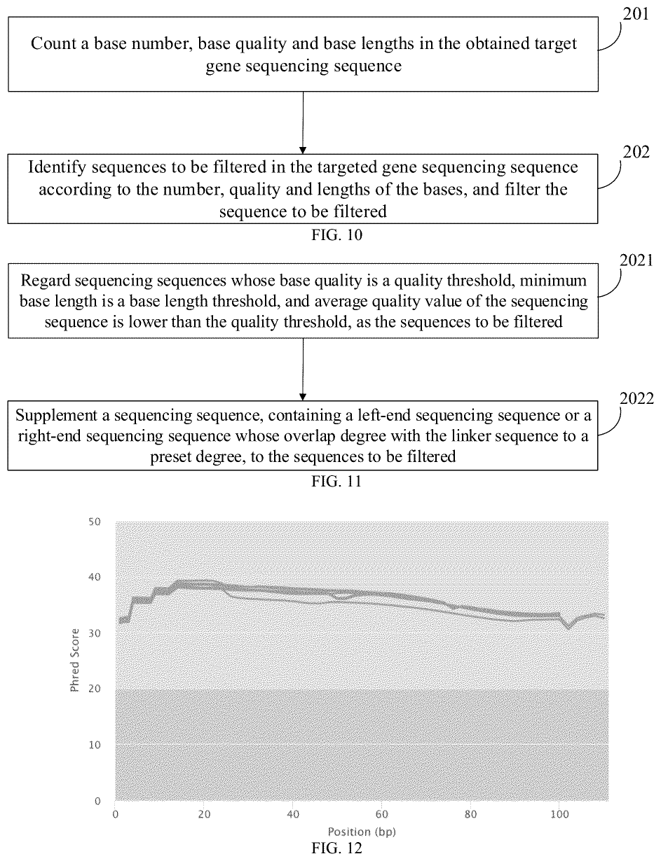

In an optional embodiment, referring to FIG. 10, before the step 101, the method further includes the following steps.
Step 201: Count a base number, base quality and base lengths in the obtained target gene sequencing sequence.

In some embodiments of the present disclosure, for the identification of linker sequences, first ten thousand or fifteen thousand lines or other first number of lines of the left-end sequencing sequences in the target gene sequencing sequence may be retrieved using a result sequence of a sequencing platform to identify a proportion of various linker sequences in the sequencing sequence, thereby determining the linker sequences used in sequencing and a proportion of the linker sequences.
Step 202: Identify sequences to be filtered in the targeted gene sequencing sequence according to the number, quality and lengths of the bases, and filter the sequences to be filtered.

In an embodiment of the present disclosure, the linker sequences are further identified specifically according to the base number, the base quality and the base lengths in the resulting sequence, thereby filtering off the linker sequences that affect the subsequent identification process, to ensure the quality of data inputted in the subsequent fusion gene identification process and improve the accuracy of the fusion gene identification.

In some embodiments, referring to FIG. 11, the step 202 includes the following steps.
Step 2021: Regard sequencing sequences whose base quality is a quality threshold, minimum base length is a base length threshold, and average quality value is lower than the quality threshold, as the sequences to be filtered.
Step 2022: supplement a sequencing sequence containing a left-end sequencing sequence or a right-end sequencing sequence whose overlap degree with the linker sequence to a preset degree, to the sequences to be filtered.

In an embodiment of the present disclosure, the data filtering criteria may be to use sequence sequences, whose base quality is equal to a base quality threshold, minimum base length is a base length threshold, and maximum sequencing error rate is an error rate threshold, as linker sequences. It is also possible to further use the sequencing sequences, of which the left-end sequencing sequence overlaps with the right-end sequencing sequence, but the length of the overlap area is greater than or equal to a preset overlap degree (for example, 3 bp) as the linker sequences. The linker sequences are excised to ensure the quality of the inputted data during subsequent fusion gene identification process and improve the accuracy of fusion gene identification.

In an exemplary embodiment, the embodiments of the present disclosure provide two examples of applying the method for identifying the fusion gene to a specific scenario for reference.

### Example I: Targeted Sequencing of Exome to Identify Gene Fusion Mutation MPRSS2-ERG

Original data is preprocessed to obtain a sequencing data volume of the original data, and the statistical results are as follows:

| Sample | Number of Reads | Number of bases |
|---|---|---|
| Sample 1-P1 | 32743311 | 3634507521 |
| Sample 1-P2 | 32743311 | 3634507521 |
| Sample 2-P1 | 67986301 | 6798630100 |
| Sample 2-P2 | 67986301 | 6798630100 |
| Sample 3-P1 | 72052839 | 7205283900 |
| Sample 3-P2 | 72052839 | 7205283900 |

The linker is detected as an illumina a sequencing platform linker: ‘AGATCGGAAGAGC’, and 2.7% of reads contain this linker. According to filtering conditions preset in S1 in the specification, the filtered data is statistically calculated as follows:

| Sample | Number of Reads | Number of bases |
|---|---|---|
| Sample 1-P1 | 28661507 | 3134253507 |
| Sample 1-P1 | 28661507 | 3055859312 |
| Sample 2-P1 | 62101252 | 6087186569 |
| Sample 2-P1 | 62101252 | 6115080856 |
| Sample 3-P1 | 64780300 | 6365445962 |
| Sample 3-P1 | 64780300 | 6335773022 |

After data processing, high-quality data is obtained, and the quality values are all above 30, referring to FIG. 12.

By aligning the filtered high-quality data to a reference genome GRCH38 and storing it in a bam format, an insert range of this group of sequencing data is calculated.

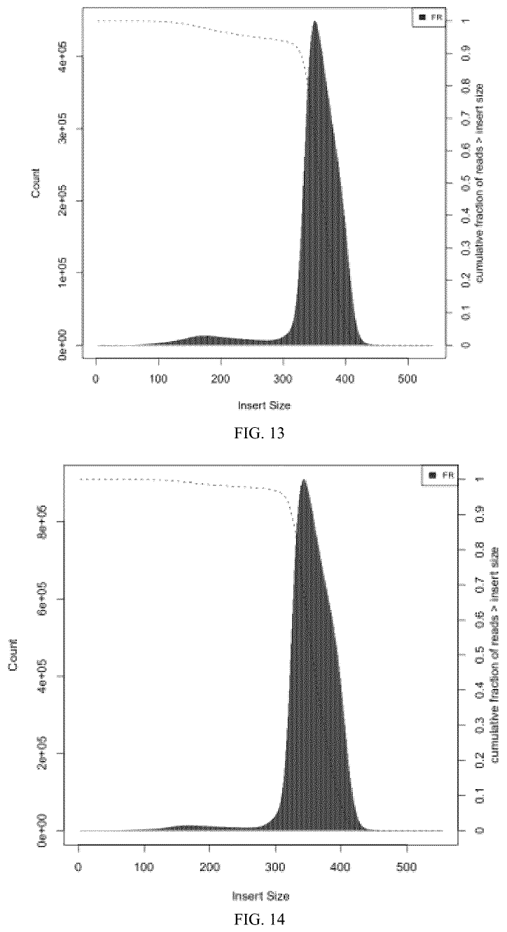

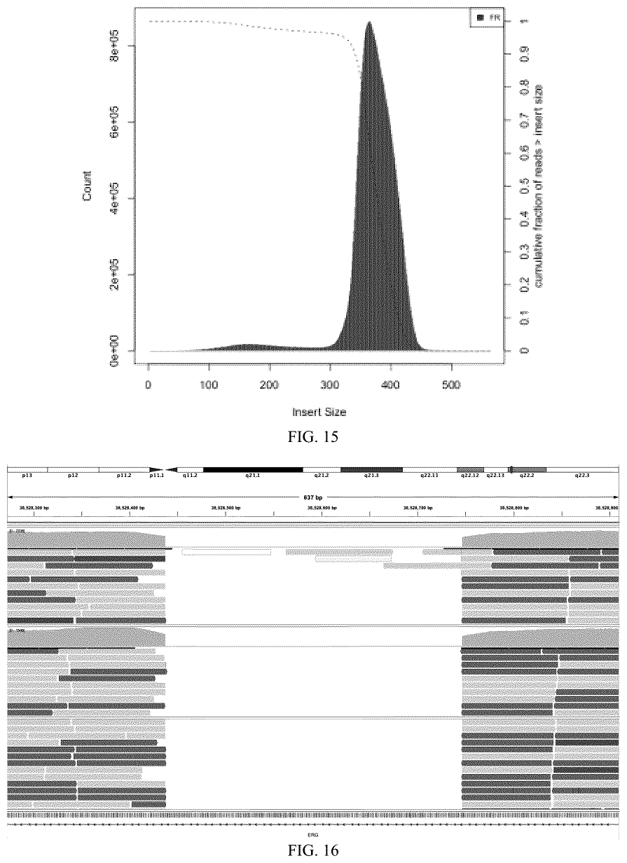

FIG. 13 shows the sample 1, FIG. 14 shows the sample 2, FIG. 15 shows the sample 3, and the insert sizes of the three samples are 359, 356, and 374, respectively.

Based on the alignment result, spanning read screening is performed.

Firstly, a distance d and lengths L1 and L2 of each mapped read are calculated, and C value parameter in Formula (1) is preset to 10.

The information of the identified spanning reads pair at least includes: mapped ReadID, an alignment Flag value of this Read, a reference sequence name, alignment to a chromosome position, an alignment quality value, an alignment match (a CIGAR string, a reference (chromosome) name obtained by alignment, an insert size in a position library mapped to the first base, a sequence fragment, and a quality value of the sequence fragment.

Based on the position information of the spanning reads and the split reads, it is identified that there are breakpoints near No. 21 chromosome at 38,528,404 and near No. 21 chromosome at 38528747. The positions are annotated with GFF3 as ERG genes in these positions. Meanwhile, there are breakpoints near No. 21 chromosome at 42,508,100 and near No. 21 at chromosome 42,508,215 bp. The positions are subjected to gene annotation as TMPRSS2 genes in these positions. Homologous gene annotation is used to prove that the two genes are not paralogous. The fusion score is calculated as 1742 according to the calculation formula of the fusion gene score, which meets the requirements of the fusion gene identification scores.

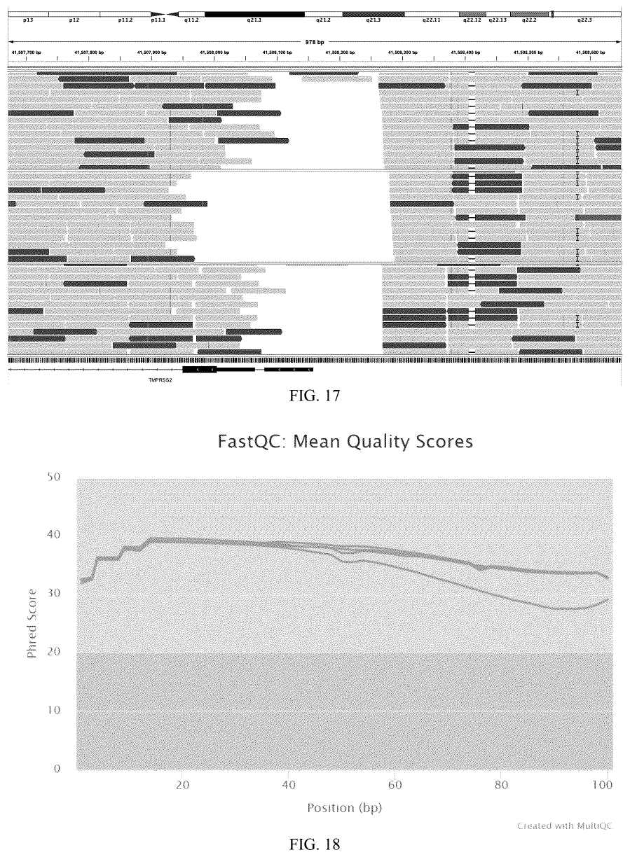

The breakpoint visualization may be referred to FIG. 16, in which the breakpoint is found on the ERG gene, and the position is between 21q22.2, 38, 528, 440 bp and 38,528,750 bp. The top, middle and bottom are sample 1, sample 2, and sample 3 in sequence: referring to FIG. 17, a breakpoint is found on the TMPRSS2 gene, and the position is between 21q22.3, 41,507,900 and 41,508,300. The top, middle and bottom are sample 1, sample 2, and sample 3 in sequence.

### Example II: Targeted Sequencing of Exome of Bladder Cancer to Identify Gene Fusion

original data is preprocessed to obtain a sequencing data volume of the original data, and the statistical results are as follows:

| Sample | Number of Reads | Number of bases |
|---|---|---|
| Sample 1-P1 | 8013317 | 801331700 |
| Sample 1-P2 | 8013317 | 801331700 |
| Sample 2-P1 | 8950252 | 895025200 |
| Sample 2-P2 | 8950252 | 895025200 |

The linker is detected as an illumina sequencing platform linker: ‘AGATCGGAAGAGC’, and 13.25% of reads contain this linker. According to filtering conditions preset in S1 in the specification, the filtered data is statistically calculated as follows:

| Sample | Number of Reads | Number of bases |
|---|---|---|
| Sample 1-P1 | 758590 | 717191169 |
| Sample 1-P2 | 758590 | 679669856 |
| Sample 2-P1 | 8603242 | 816311390 |
| Sample 2-P2 | 8603242 | 817245409 |

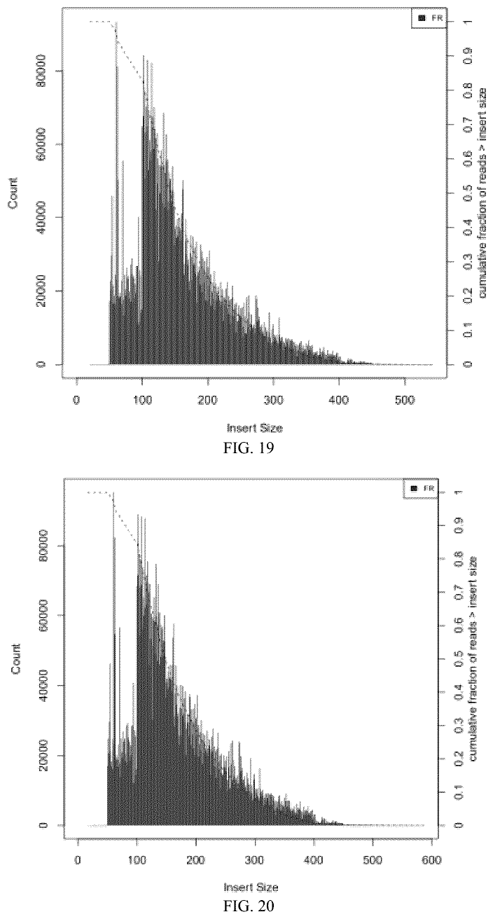

After data processing, high-quality data is obtained, and the quality values of the bases of two samples are both above 30, and an average value of some bases between 80 to 100 bp is close to a critical value of 30, referring to FIG. 18. By aligning the filtered high-quality data to the reference genome GRCH38 and storing it in a bam format, an insert range of this group of sequencing data is calculated. The insert sizes of the two samples are 142, 147 respectively. FIG. 19 shows the sample 1, and FIG. 20 shows the sample 2.

Because no breakpoints satisfying both spanning reads and split reads are identified, no gene fusion is present in the sample data.

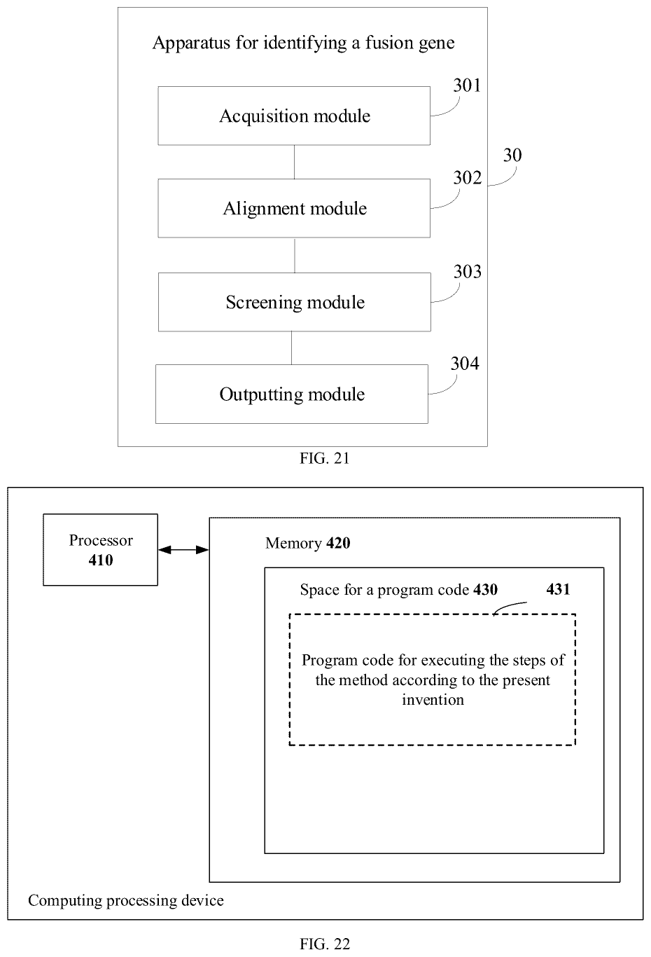

FIG. 21 schematically shows a schematic structural diagram of an apparatus 30 for identifying a fusion gene according to the present disclosure. The apparatus includes:
an acquisition module 301 configured to acquire a target gene sequencing sequence to be identified and a reference gene sequence;
an alignment module configured to align the sequencing sequence of the target gene to the reference gene sequence, and acquire distribution and targeted capturing results of spanning reads and split reads of the target gene sequencing sequence located in a target area;
a screening module 303 configured to screen a target fusion gene pair from the reads based on the distribution and targeted capture results; and an outputting module 304 configured to output an identification result regarding the target fusion gene pair.

In an optional embodiment, the screening module 303 is configured to:
calculate breakpoint positions of the reads according to the number of the split reads and the number of strongly supported split reads;
screen a target fusion gene pair from the reads according to the breakpoint positions and the positions of the spanning reads.

In an optional embodiment, the screening module 303 is further configured to:
filter a read that does not have the supported spanning read, at the upstream and downstream of a breakpoint position included in the read;
regard the read reserved after filtration as candidate fusion gene pairs in the case that
the first end and the second end of the reads reserved after filtration are located in different genes; and
filter a low-quality fusion gene pair in the candidate fusion gene pairs to obtain the target fusion gene pair.

In an optional embodiment, the screening module 303 is further configured to:
filter paralogous genes in the candidate fusion gene pairs to obtain first candidate fusion gene pairs;
calculate the number of gene mappings contained in the first candidate fusion genes;
filter the first candidate fusion gene pairs in which the number of the gene mappings is greater than or equal to a number threshold of gene mappings to obtain second candidate fusion gene pairs;
calculate a fusion gene score of the second candidate fusion gene pair according to a distance between the breakpoint positions of the second candidate fusion gene pair, and an average sequencing depth in the target area; and
filter the second candidate fusion gene pair whose fusion gene score is less than a fusion gene score threshold to obtain the target candidate fusion gene pair.

In an optional embodiment, the screening module 303 is further configured to:
solve a difference between a sum of the distances between the spanning read in the second fusion gene pairs and two breakpoints and a peak value of an insert length of the genome methylated sequencing sequence, as a first factor score;
regard a ratio of distances between two ends of the spanning read in the second fusion gene pairs and the breakpoint position to a length of the read as a second factor score;
regard a ratio of distances between two ends of the split read in the second fusion gene pairs and the breakpoint position to a multiplication length of the read as a third factor score, the multiplication length being a product of the length of the sequenced fragment and a multiplication parameter; and
regard a ratio of a sum of the first factor score, the second factor score and the third factor score to the average sequencing depth in the target area as a fusion gene score of the second candidate fusion gene pairs.

In an optional embodiment, the alignment module 302 is further configured to:
align the target gene sequencing sequence to the reference gene sequence to obtain an alignment result; and
screen the spanning reads from the targeted sequencing result based on the alignment result in spanning read screening conditions, and screen the split reads and the strongly supported split reads from the targeted sequencing result based on the alignment result in split read screening conditions.

In an optional embodiment, the alignment module 302 is further configured to:
screen a spanning read that meets the following spanning read screening conditions at the same time from the reads:
a sum value obtained by summing a length of the left-end read, a length of the right-end
read and a distance between the left-end read and the right-end read of the read is greater than a product of the lower quartile of the length of the read and a target parameter, the target parameter being a parameter that controls the number of outputted mappings and the degree of stringency;
neither the left-end read nor the right-end read in the reads has a similar sequence; and
multiple alignment values of the left-end read and the right-end read in the reads include a proper aligner characteristic value and a secondary alignment characteristic value, and does not include a segment unmapped characteristic value and a next segment unmapped characteristic value.

In an optional embodiment, the alignment module 302 is further configured to:
screen a split read that meets the following split read screening conditions at the same time from the reads:
an alignment length of each position in the read is greater than a length threshold, and the alignment length is greater than one-third of a total length of the read;
a sequence whose length of the read is the alignment length has no similar sequence in the alignment result; and
a quality value of the number of alignment times of the read is greater than or equal to a number of alignment times; and
determine the split read that meet the above split read conditions, in the case that alignment positions of the left-end read and the right-end read overlap, to be a strongly supported split read.

In an optional embodiment, the screening module 303 is further configured to:
regard a maximum value of a weighted sum of the number of split reads in the reads and the number of the strongly supported split reads as a breakpoint position.

In an optional embodiment, the acquisition module 301 is further configured to:
count a base number, base quality and base lengths in the obtained target gene sequencing sequence; and
identify sequences to be filtered in the targeted gene sequencing sequence according to the base number, the base quality and the base lengths, and filter the sequences to be filtered.

In an optional embodiment, the acquisition module 301 is further configured to:
regard sequencing sequences whose base quality is a quality threshold, minimum base length is a base length threshold, and average quality value of the sequencing sequence is lower than the quality threshold, as the sequences to be filtered; and
supplement a sequencing sequence containing a left-end sequencing sequence or a right-end sequencing sequence whose overlap degree with the linker sequence reaching a preset degree, to the sequences to be filtered.

In an embodiment of the present disclosure, by performing fusion gene identification for conventional targeted sequencing and aligning the reference gene sequence to the target area of targeted sequencing to obtain the distribution and target capture results of the split reads and spanning reads near the target area. The fusion gene pairs in the target gene sequencing sequence are screened out by using the distribution characteristics of the split reads and the spanning reads in the fusion gene pairs. The screening may be performed against the distribution of reads of different fusion genes, so that the fusion gene identification results of the targeted gene sequencing sequence are no longer limited to specific fusion gene species, which increases the utilization rate of targeted gene sequencing data in gene fusion identification.

The various component embodiments of the present disclosure can be implemented in hardware, software modules running on one or more processors, or their combination. Those skilled in the art should understand that a microprocessor or digital signal processor (DSP) can be used in practice to realize some or all functions of some or all components of the computing processing device according to the embodiment of the present disclosure. The present disclosure may also be implemented as a device or device program (e. g., a computer program and a computer program product) for performing part or all of the methods described herein. Such a program to realize the present disclosure can be stored on a non-transient computer-readable medium, or can have the form of one or more signals. Such signals can be downloaded from Internet websites, or provided on carrier signals, or in any other form.

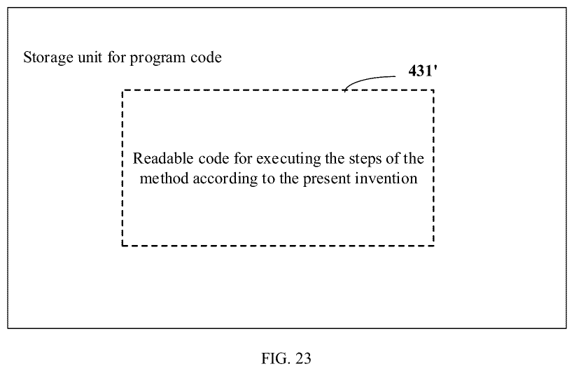

For example, FIG. 22 shows a computing processing device that can implement the method according to the present disclosure. The computing processing device traditionally includes a processor 410 and a computer program product in the form of a memory 420 or a non-transient computer-readable medium. The memory 420 may be an electronic memory such as flash memory, electrically erasable programmable read-only memory (EEPROM), EPROM, hard disk, or ROM. The memory 420 has a storage space 430 for program code 431 for executing any of the above method steps. For example, the storage space 430 for program code may include individual program codes 431 for implementing various steps in the above method. These program codes can be read from or written into one or more computer program products. These computer program products include program code carriers such as hard disks, compact discs (CDs), memory cards or floppy disks. Such computer program products are usually portable or fixed storage units as described with reference to FIG. 23. The storage unit may have storage segments, storage space, and the like arranged similarly to the memory 420 in the computing processing apparatus of FIG. 22. The program code can be compressed in an appropriate form, for example. Generally, the storage unit includes computer-readable code 431′, that is, code that can be read by a processor such as 410, which, when run by the computing processing device, causes the computing processing device to perform various steps in the method described above.

It should be understood that although each step in the flow chart of the attached drawings is displayed in sequence according to the arrow, these steps are not necessarily executed in sequence according to the arrow: Unless explicitly stated in the specification, the execution of these steps is not strictly limited in order, and they can be executed in other order. Moreover, at least one part of the steps in the flow chart of the attached drawing may include multiple sub-steps or stages. These sub-steps or stages may not necessarily be completed at the same time, but may be executed at different times, and the execution order may not necessarily be sequential, but may be executed alternately or alternately with other steps or sub-steps or at least part of other steps or stages.

The “one embodiment”, “embodiments” or “one or more embodiments” mentioned herein means that the specific features, structures or features described in combination with the embodiments are included in at least one embodiment of the present disclosure. In addition, please note that the word “in one embodiment” does not necessarily refer to the same embodiment.

A large number of specific details are described in the instructions provided here. However, it can be understood that the embodiments of the present disclosure can be practiced without these specific details. In some examples, the well-known methods, structures and techniques are not shown in detail so as not to obscure the understanding of this specification.

In the claims, any reference symbol between brackets shall not be constructed as a restriction on the claims. The word “comprising” does not exclude the existence of elements or steps not listed in the claims. The word “a/an” or “one” before a component does not exclude the existence of multiple such components. The present disclosure can be realized by means of hardware including several different elements and by means of a properly programmed computer. In the unit claims that list several devices, several of these devices can be embodied by the same hardware item. The use of the first, second, and third words does not indicate any order. These words can be interpreted as names.

Finally, it should be noted that the above embodiments are only used to illustrate the technical solution of the present disclosure, not to limit it. Although the present disclosure has been described in detail with reference to the preceding embodiments, those skilled in the art should understand that they can still modify the technical solutions recorded in the preceding embodiments or replace some of the technical features equally. These modifications or substitutions do not make the essence of the corresponding technical solutions separate from the spirit and scope of the technical solutions of the embodiments of the present disclosure.

1. A method for identifying a fusion gene, comprising:
acquiring a target gene sequencing sequence to be identified and a reference gene sequence;
aligning the target gene sequencing sequence to the reference gene sequence, and acquiring distribution and targeted capturing results of spanning reads and split reads of the target gene sequencing sequence located in a target area;
screening a target fusion gene pair from the reads based on the distribution and the targeted capture results; and
outputting an identification result regarding the target fusion gene pair.

2. The method according to claim 1, wherein the step of screening the target fusion gene pair from the reads based on the distribution and the targeted capture results comprises:
calculating a breakpoint position of the read according to the number of the split reads and the number of strongly supported split reads; and
screening the target fusion gene pair from the reads according to the breakpoint position and positions of the spanning reads.

3. The method according to claim 2, wherein the step of screening the target fusion gene pair from the read according to the breakpoint position and the positions of the spanning reads comprises:
filtering a read that does not have the supported spanning read, at the upstream and downstream of the breakpoint position included in the read;
regarding the reads reserved after filtration as candidate fusion gene pairs in the case that the first end and the second end of the read reserved after filtration are located in different genes; and
filtering a low-quality fusion gene pair in the candidate fusion gene pairs to obtain the target fusion gene pair.

4. The method according to claim 3, wherein the step of filtering the low-quality fusion gene pair in the candidate fusion gene pairs to obtain the target fusion gene pair comprises:
filtering paralogous genes in the candidate fusion gene pairs to obtain first candidate fusion gene pairs;
calculating a number of gene mappings contained in the first candidate fusion genes;
filtering the first candidate fusion gene pairs in which the number of the gene mappings is greater than or equal to a number threshold of gene mappings to obtain a second candidate fusion gene pair;
calculating a fusion gene score of the second candidate fusion gene pair according to a distance between the breakpoint positions of the second candidate fusion gene pair, and an average sequencing depth in the target area; and
filtering the second candidate fusion gene pair whose fusion gene score is less than a fusion gene score threshold to obtain the target candidate fusion gene pair.

5. The method according to claim 4, wherein the step of calculating the fusion gene score of the second candidate fusion gene pair according to the distance between the breakpoint positions of the second candidate fusion gene pair, and the average sequencing depth in the target area, comprises:
solving a difference between a sum of the distances between the spanning read in the second fusion gene pair and two breakpoints, and a peak value of an insert length of a genome methylated sequencing sequence, as a first factor score;
regarding a ratio of distances between two ends of the spanning read in the second fusion gene pair and the breakpoint position to a length of the read as a second factor score;
regarding a ratio of distances between two ends of the split read in the second fusion gene pair and the breakpoint position to a multiplication length of the read as a third factor score, wherein the multiplication length is a product of the length of the read and a multiplication parameter; and
regarding a ratio of a sum of the first factor score, the second factor score and the third factor score to the average sequencing depth in the target area as the fusion gene score of the second candidate fusion gene pair.

6. The method according to claim 2, wherein the step of aligning the target gene sequencing sequence to the reference gene sequence, and acquiring the distribution and targeted capturing results of the spanning reads and split reads of the target sequencing sequence located in the target area comprises:
aligning the target gene sequencing sequence to the reference gene sequence to obtain an alignment result; and
screening the spanning reads from the targeted sequencing result based on the alignment result in spanning read screening conditions, and screening the split reads and the strongly supported split reads from the targeted sequencing result based on the alignment result in split read screening conditions.

7. The method according to claim 6, wherein the step of screening the spanning reads from the targeted sequencing result based on the alignment result in the spanning read screening conditions comprises:
screening a spanning read that meets the following spanning read screening conditions at the same time from the reads:
a sum value obtained by summing a length of a left-end read, a length of a right-end read and a distance between the left-end read and the right-end read of the read is greater than a product of the lower quartile of a length of the read and a target parameter, wherein the target parameter is a parameter that controls a number of outputted mappings and degree of stringency;
neither the left-end read nor the right-end read in the reads has a similar sequence; and
multiple alignment values of the left-end read and the right-end read in the reads comprise a proper aligner characteristic value and a secondary alignment characteristic value, and does not include a segment unmapped characteristic value and a next segment unmapped characteristic value.

8. The method according to claim 6, wherein the step of screening the split reads and the strongly supported split reads in the targeted sequencing result based on the alignment result in the split read screening conditions, comprises:
screening a split read that meets the following split read screening conditions at the same time from the reads:
an alignment length of each position in the read is greater than a length threshold, and the alignment length is greater than one-third of a total length of the read;
a sequence whose length of the read is the alignment length has no similar sequence in the alignment result; and
a quality value of a number of alignment times of the read is greater than or equal to a number of alignment times; and
determining the split read that meets the above split read conditions, in the case that alignment positions of the left-end read and the right-end read overlap, to be a strongly supported split read.

9. The method according to claim 2, wherein the step of calculating the breakpoint position of the read according to the number of the split reads and the number of the strongly supported split reads comprises:
regarding a maximum value of a weighted sum of the number of the split reads and the number of the strongly supported split reads in the reads as the breakpoint position.

10. The method according to claim 1, wherein, before acquiring the target gene sequencing sequence to be identified and the reference gene sequence, the method further comprises:
counting a base number, base quality and base lengths in the obtained target gene sequencing sequence; and
identifying sequences to be filtered in the targeted gene sequencing sequence according to the base number, the base quality and the base lengths, and filtering the sequences to be filtered.

11. The method according to claim 10, wherein the step of identifying the sequences to be filtered in the target gene sequencing sequence according to the number of the bases, the quality of the bases and the lengths of the bases comprises:
regarding sequencing sequences whose base quality is a quality threshold, minimum base length is a base length threshold, and average quality value of the sequencing sequence is lower than the quality threshold, as the sequences to be filtered; and
supplementing a sequencing sequence containing a left-end sequencing sequence or a right-end sequencing sequence whose overlap degree with the sequences to be filtered reaching a preset degree, to the sequences to be filtered.

12. (canceled)

13. A computing processing device, comprising:
a memory, configured to store a computer-readable code therein; and
one or more processors, when the computer-readable code is executed by the one or more processors, causes the computing processing device to execute operations according to claim 1.

14. A computer program product, comprising a computer-readable code, which when being operated on the computing processing device, causes the computing processing device to execute operations according to claim 1.

15. A non-transitory computer-readable medium, storing a computer program therein for performing operations according to claim 1.

16. The computing processing device according to claim 13, wherein the operation of screening the target fusion gene pair from the reads based on the distribution and the targeted capture results comprises:
calculating a breakpoint position of the read according to the number of the split reads and the number of strongly supported split reads; and
screening the target fusion gene pair from the reads according to the breakpoint position and positions of the spanning reads.

17. The computing processing device according to claim 16, wherein the operation of screening the target fusion gene pair from the read according to the breakpoint position and the positions of the spanning reads comprises:
filtering a read that does not have the supported spanning read, at the upstream and downstream of the breakpoint position included in the read;
regarding the reads reserved after filtration as candidate fusion gene pairs in the case that the first end and the second end of the read reserved after filtration are located in different genes; and
filtering a low-quality fusion gene pair in the candidate fusion gene pairs to obtain the target fusion gene pair.

18. The computer program product according to claim 14, wherein the operation of screening the target fusion gene pair from the reads based on the distribution and the targeted capture results comprises:
calculating a breakpoint position of the read according to the number of the split reads and the number of strongly supported split reads; and
screening the target fusion gene pair from the reads according to the breakpoint position and positions of the spanning reads.

19. The computer program product according to claim 18, wherein the operation of screening the target fusion gene pair from the read according to the breakpoint position and the positions of the spanning reads comprises:
filtering a read that does not have the supported spanning read, at the upstream and downstream of the breakpoint position included in the read;
regarding the reads reserved after filtration as candidate fusion gene pairs in the case that the first end and the second end of the read reserved after filtration are located in different genes; and
filtering a low-quality fusion gene pair in the candidate fusion gene pairs to obtain the target fusion gene pair.

20. The non-transitory computer-readable medium according to claim 15, wherein the operation of screening the target fusion gene pair from the reads based on the distribution and the targeted capture results comprises:
calculating a breakpoint position of the read according to the number of the split reads and the number of strongly supported split reads; and
screening the target fusion gene pair from the reads according to the breakpoint position and positions of the spanning reads.

21. The non-transitory computer-readable medium according to claim 20, wherein the operation of screening the target fusion gene pair from the read according to the breakpoint position and the positions of the spanning reads comprises:
filtering a read that does not have the supported spanning read, at the upstream and downstream of the breakpoint position included in the read;
regarding the reads reserved after filtration as candidate fusion gene pairs in the case that the first end and the second end of the read reserved after filtration are located in different genes; and
filtering a low-quality fusion gene pair in the candidate fusion gene pairs to obtain the target fusion gene pair.
